(19) United States
(12) Patent Application Publication
(10) Pub. No.: US 2024/0226575 A1
(43) Pub. Date: Jul. 11, 2024

(54) RHYTHM DISCRIMINATION WITH THREE-AXIS ACCELEROMETER IN AN IMPLANTED MEDICAL DEVICE

(71) Applicant: CARDIAC PACEMAKERS, INC., St. Paul, MN (US)

(72) Inventors: Steven Lee Eddy, Chesterfield, VA (US); Michael J. Kane, St. Paul, MN (US); Bryan J. Swackhamer, Shoreview, MN (US)

(73) Assignee: CARDIAC PACEMAKERS, INC., St. Paul, MN (US)

(21) Appl. No.: 18/404,089

(22) Filed: Jan. 4, 2024

Related U.S. Application Data
(60) Provisional application No. 63/437,446, filed on Jan. 6, 2023.

(51) Int. Cl. A61N 1/365; A61N 1/37; A61N 1/375

(52) U.S. Cl. CPC A61N 1/36585; A61N 1/3702; A61N 1/3756

(57) ABSTRACT

A leadless cardiac pacemaker (LCP) may include a three-axis accelerometer. Acceleration signals from each of the three axes of the accelerometer may be combined into a combined acceleration signal and a predetermined morphological feature may be identified in a magnitude of the combined acceleration signal. The relative timing of the predetermined morphological signal relative to the cardiac cycle duration may be used to ascertain whether a detected arrhythmia is an arrhythmia that should be treated by the LCP or should not be treated by the LCP.

### CROSS REFERENCE TO RELATED APPLICATIONS

The present application claims the benefit of and priority to US Provisional Patent Application No. 63/437,446, filed Jan. 6, 2023, the disclosure of which is incorporated herein by reference.

### TECHNICAL FIELD

The present disclosure generally relates to systems, devices, and methods for detecting arrhythmia or confirming detection of arrhythmia of a heart of a patient, and more particularly to systems, devices, and methods for detecting arrhythmia or confirming detection of arrhythmia of a heart using a three-axis accelerometer

### BACKGROUND

Pacing instruments can be used to treat patients suffering from various heart conditions that result in a reduced ability of the heart to deliver sufficient amounts of blood to a patient's body. These heart conditions may lead to rapid, irregular, and/or inefficient heart contractions. To help alleviate some of these conditions, various devices (e.g., pacemakers, defibrillators, etc.) can be implanted in a patient's body. Such devices may monitor and provide electrical stimulation to the heart to help the heart operate in a more normal, efficient and/or safe manner. In some cases, the devices may be part of an implantable medical device system.

### SUMMARY

The present disclosure generally relates to systems, devices, and methods for detecting arrhythmia or confirming detection of arrhythmia of a heart of a patient, and more particularly to systems, devices, and methods for detecting arrhythmia or confirming detection of arrhythmia of a heart using a three-axis accelerometer. An example may be found in a leadless cardiac pacemaker (LCP) for implantation in a heart of a patient. The LCP includes a housing, two or more electrodes exposed to an exterior of the housing, a three-axis accelerometer disposed within the housing, the three-axis accelerometer providing an acceleration signal for each of the three axes of the three-axis accelerometer, a memory and a controller disposed within the housing and operably coupled with the two or more electrodes, the three-axis accelerometer and the memory. The controller is configured to identify a cardiac cycle of the heart, the cardiac cycle having a cardiac cycle duration that is dependent on a current heart rate of the heart, and determine whether the heart is experiencing an arrhythmia. The controller is configured to receive the acceleration signal from each of the three axes of the three-axis accelerometer, combine the acceleration signals from each of the three axes of the three-axis accelerometer into a combined acceleration signal having a magnitude, and identify a predetermined morphological feature in the magnitude of the combined acceleration signal. The controller is configured to identify a relative time of occurrence of the predetermined feature within the cardiac cycle duration and to determine whether the arrhythmia is an arrhythmia that should be treated by delivery of a therapy by the LCP or an arrhythmia that should not be treated by the LCP based at least in part on the relative time of occurrence of the predetermined feature within the cardiac cycle duration. When the arrhythmia is determined to be an arrhythmia that should be treated by delivery of a therapy by the LCP, the controller is configured to deliver the therapy to the heart via two or more electrodes of the LCP. When the arrhythmia is determined to be an arrhythmia that should not be treated by the LCP, the controller is configured to not deliver the therapy to the heart via two or more electrodes of the LCP.

Alternatively or additionally, the controller may be configured to combine the acceleration signals into the combined acceleration signal by one or more of calculating a sum of the acceleration signals from each of the three axes of the three-axis accelerometer, calculating a root mean square of the acceleration signals from each of the three axes of the three-axis accelerometer, or calculating a root sum square of the acceleration signals from each of the three axes of the three-axis accelerometer into the combined acceleration signal.

Alternatively or additionally, the controller may be configured to receive an electrocardiogram signal via the two or more electrodes that are exposed to the exterior of the housing and to identify the cardiac cycle of the heart based at least in part on the electrocardiogram signal.

Alternatively or additionally, the controller may be configured to determine whether the heart is experiencing an arrhythmia based at least in part on the electrocardiogram signal.

Alternatively or additionally, the controller may be configured to determine whether the heart is experiencing an arrhythmia based at least in part on the acceleration signal of one or more of the three axes of the three-axis accelerometer.

Alternatively or additionally, the controller may be configured to determine whether the arrhythmia is an arrhythmia that should be treated by delivery of the therapy by the LCP or an arrhythmia that should not be treated by the LCP based at least in part on whether the relative time of occurrence of the predetermined feature falls within a defined time window of the cardiac cycle duration, wherein the defined time window has a duration that is less than the cardiac cycle duration.

Alternatively or additionally, the controller may be configured to determine that the arrhythmia is an arrhythmia that should be treated by delivery of the therapy by the LCP when the relative time of occurrence of the predetermined feature falls within the defined time window of the cardiac cycle duration of the cardiac cycle.

Alternatively or additionally, the defined time window may have a duration that corresponds to a first predetermined percentage of the cardiac cycle duration and a start time that corresponds to a second predetermined percentage of the cardiac cycle duration.

Alternatively or additionally, once the controller determines whether the arrhythmia is an arrhythmia that should be treated by delivery of a therapy by the LCP or an arrhythmia that should not be treated by the LCP, the controller may be configured to communicate that determination to one or more other devices.

Alternatively or additionally, the controller may be configured to express a relationship between the cardiac cycle duration and the magnitude of the combined acceleration signal using a polar notation, where the cardiac cycle duration is normalized to 2π radians or 360 degrees, and the relative time of occurrence of the predetermined feature within the cardiac cycle duration is determined by an angular direction at which the predetermined feature is observed in the polar notation.

Alternatively or additionally, the predetermined morphological feature may be one or more of a minimum in the magnitude of the combined acceleration signal, a maximum in the magnitude of the combined acceleration signal, a maximum change versus time of the magnitude of the combined acceleration signal, and a maximum change in the change versus time of the magnitude of the combined acceleration signal.

Alternatively or additionally, the controller may identify the predetermined morphological feature by comparing the magnitude of the combined acceleration signal with a plurality of morphological feature templates, and may identify the predetermined morphological feature as that which corresponds to a matching one of the plurality of morphological feature templates.

Alternatively or additionally, the controller may be configured to identify the relative time of occurrence of the predetermined feature within the cardiac cycle duration for each of a plurality of cardiac cycles of the heart, determine a variability in the relative time of occurrence of the predetermined feature within the cardiac cycle duration among the plurality of cardiac cycles of the heart, and determine whether the arrhythmia is an arrhythmia that should be treated by delivery of a therapy by the LCP or an arrhythmia that should not be treated by the LCP based at least in part on the relative time of occurrence of the predetermined feature within the cardiac cycle duration and the variability in the relative time of occurrence of the predetermined feature within the cardiac cycle duration for each of the plurality of cardiac cycles of the heart.

Alternatively or additionally, the controller may be further configured to determine a posture of the patient based at least in part on the acceleration signal of one or more of the three axes of the three-axis accelerometer. The controller may be configured to determine whether the arrhythmia is an arrhythmia that should be treated by delivery of a therapy by the LCP or an arrhythmia that should not be treated by the LCP based at least in part on the relative time of occurrence of the predetermined feature within the cardiac cycle duration and the posture of the patient.

Another example may be found in a method for operating an implantable medical device (IMD) for implantation in a heart of a patient, the IMD having a three-axis accelerometer that provides an acceleration signal for each of the three axes of the three-axis accelerometer. The method includes identifying a cardiac cycle of the heart, the cardiac cycle having a cardiac cycle duration that is dependent on a current heart rate of the heart, combining the acceleration signals from each of the three axes of the three-axis accelerometer into a combined acceleration signal having a magnitude, identifying a predetermined morphological feature in the magnitude of the combined acceleration signal, and identifying a relative time of occurrence of the predetermined feature within the cardiac cycle duration. The controller is configured to discriminate between two or more different arrhythmia types based at least in part on the relative time of occurrence of the predetermined feature within the cardiac cycle duration. The IMD causes delivery of a therapy to the heart (by the IMD and/or another device) when an arrhythmia of a first arrhythmia type is identified and the IMD not causing delivery of the therapy (by the IMD and/or another device) to the heart when an arrhythmia of a second arrhythmia type is identified.

Alternatively or additionally, discriminating between two or more different arrhythmia types may be based at least in part on whether the relative time of occurrence of the predetermined feature falls within a defined time window of the cardiac cycle duration, wherein the defined time window has a duration that is less than the cardiac cycle duration.

Alternatively or additionally, the arrhythmia may be identified as the first arrhythmia type when the relative time of occurrence of the predetermined feature falls within the defined time window of the cardiac cycle duration of the cardiac cycle.

Alternatively or additionally, the method may include identifying the relative time of occurrence of the predetermined feature within the cardiac cycle duration for each of a plurality of cardiac cycles of the heart, determining a variability in the relative time of occurrence of the predetermined feature within the cardiac cycle duration among the plurality of cardiac cycles of the heart and discriminating between two or more different arrhythmia types based at least in part on the relative time of occurrence of the predetermined feature within the cardiac cycle duration and the variability in the relative time of occurrence of the predetermined feature within the cardiac cycle duration for each of the plurality of cardiac cycles of the heart.

Another example may be found in a non-transitory computer readable medium storing instructions that when executed by one or more processors cause the one or more processors to identify a cardiac cycle of a heart, the cardiac cycle having a cardiac cycle duration that is dependent on a current heart rate of the heart. The one or more processors are caused to combine acceleration signals from each of three axes of a three-axis accelerometer into a combined acceleration signal having a magnitude. The one or more processors are caused to identify a predetermined morphological feature in the magnitude of the combined acceleration signal. The one or more processors are caused to identify a relative time of occurrence of the predetermined feature within the cardiac cycle duration. The one or more processors are caused to discriminate between two or more different arrhythmia types based at least in part on the relative time of occurrence of the predetermined feature within the cardiac cycle duration. The one or more processors are caused to cause delivery of a therapy to the heart when an arrhythmia of a first arrhythmia type is identified and not cause delivery of the therapy to the heart when an arrhythmia of a second arrhythmia type is identified.

Alternatively or additionally, the one or more processors are caused to discriminate between two or more different arrhythmia types based at least in part on whether the relative time of occurrence of the predetermined feature falls within a defined time window of the cardiac cycle duration, wherein the defined time window has a duration that is less than the cardiac cycle duration.

The above summary is not intended to describe each embodiment or every implementation of the present disclosure. Advantages and attainments, together with a more complete understanding of the disclosure, will become apparent and appreciated by referring to the following description and claims taken in conjunction with the accompanying drawings.

### BRIEF DESCRIPTION OF THE DRAWINGS

The disclosure may be more completely understood in consideration of the following description of various illustrative embodiments in connection with the accompanying drawings, in which:

FIG. 1 is a schematic block diagram of an illustrative leadless cardiac pacemaker (LCP);

FIG. 2 is a flow diagram showing an illustrative method for operating an implantable medical device (IMD) such as the LCP of FIG. 1;

FIGS. 3A and 3B are flow diagrams that together show an illustrative method for operating an implantable medical device (IMD) such as the LCP of FIG. 1;

FIG. 4 is another schematic block diagram of an illustrative leadless cardiac pacemaker (LCP);

FIG. 5 is a schematic block diagram of illustrative medical device that may be used in conjunction with the LCP of FIG. 4;

FIG. 6 is a schematic diagram of an illustrative medical system that includes multiple LCPs and/or other devices in communication with one another;

FIG. 7 is a schematic diagram of an illustrative system including an LCP and another medical device, in accordance with another embodiment of the present disclosure; and

FIGS. 8 through 10 are graphs of illustrative canine intracardiac accelerometer data plotted in polar notation versus cardiac cycle phase angle in degrees.

While the disclosure is amenable to various modifications and alternative forms, specifics thereof have been shown by way of embodiment in the drawings and will be described in detail. It should be understood, however, that the intention is not to limit aspects of the disclosure to the particular illustrative embodiments described. On the contrary, the intention is to cover all modifications, equivalents, and alternatives falling within the spirit and scope of the disclosure.

### DESCRIPTION

The following description should be read with reference to the drawings in which similar elements in different drawings are numbered the same. The description and the drawings, which are not necessarily to scale, depict illustrative embodiments and are not intended to limit the scope of the disclosure.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

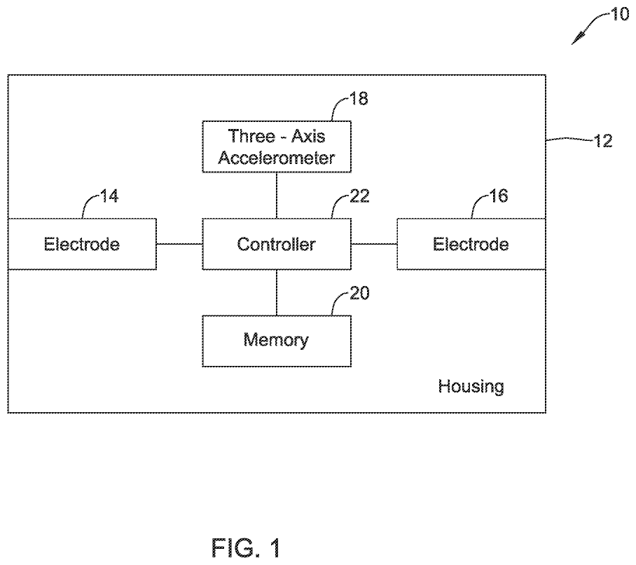

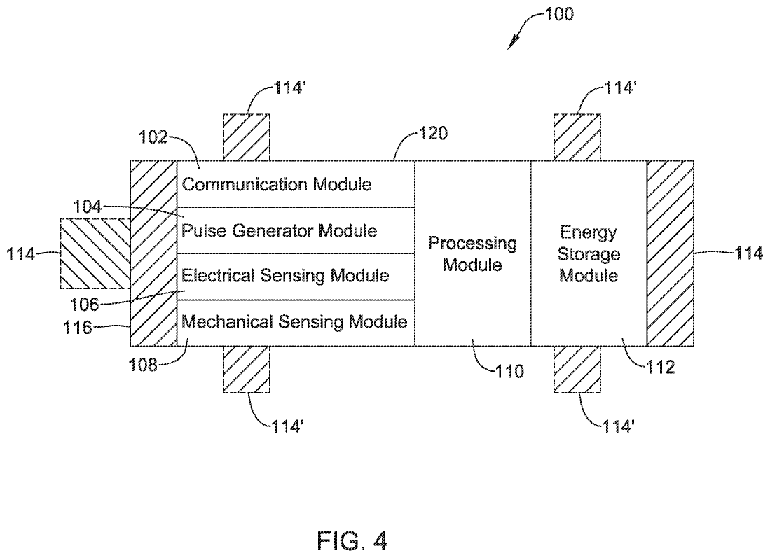

FIG. 1 is a schematic block diagram of an illustrative leadless cardiac pacemaker (LCP) 10 that is configured for implantation within or on a heart of a patient. The illustrative LCP 10 includes a housing 12 and at least two electrodes 14 and 16 that are exposed to an exterior of the housing 12. While two electrodes 14 and 16 are shown, this is merely illustrative, as the LCP 10 may include three, four or more electrodes exposed to the exterior of the housing 12. A three-axis accelerometer 18 is disposed within the housing 12 and is configured to provide an acceleration signal for each of the three axes of the three-axis accelerometer. The LCP 10 includes a memory 20 and a controller 22 that is disposed within the housing 12 and that is operably coupled with the electrodes 14 and 16. It will be appreciated that the LCP 10 may include additional components not expressly illustrated, such as but not limited to an energy storage module (see, for example, FIG. 4).

In the example shown, the controller 22 is configured to identify a cardiac cycle of the heart, the cardiac cycle having a cardiac cycle duration that is dependent on a current heart rate of the heart. In some cases, the controller 22 is configured to receive an electrocardiogram signal via the electrodes 14 and 16, and to identify the cardiac cycle of the heart based at least in part on the electrocardiogram signal. In some cases, the controller 22 is configured to identify the cardiac cycle of the heart based at least in part on the acceleration signal of one or more of the three axes of the three-axis accelerometer 18. In some cases, the controller 22 is configured to identify the cardiac cycle of the heart based at least in part on the electrocardiogram signal and one or more of the three axes of the three-axis accelerometer 18. These are just examples.

The controller 22 is configured to determine whether the heart is experiencing an arrhythmia. In some cases, the controller 22 may be configured to determine whether the heart is experiencing an arrhythmia based at least in part on the electrocardiogram signal. In some cases, the controller 22 may be configured to determine whether the heart is experiencing an arrhythmia based at least in part on the acceleration signal of one or more of the three axes of the three-axis accelerometer 18. In some cases, the controller 22 may be configured to determine whether the heart is experiencing an arrhythmia based at least in part on the electrocardiogram signal and the acceleration signal of one or more of the three axes of the three-axis accelerometer 18. In some cases, the controller 22 may be configured to determine whether the heart is experiencing an arrhythmia based at least in part on a communication received from a remote device, such as an SICD (Subcutaneous Implantable Cardioverter-Defibrillator) or other device.

The controller 22 is configured to receive the acceleration signal from each of the three axes of the three-axis accelerometer 18, combine the acceleration signals from each of the three axes of the three-axis accelerometer into a combined acceleration signal having a magnitude, and identify a predetermined morphological feature in the magnitude of the combined acceleration signal. In some cases, the predetermined morphological feature may include one or more of a minimum in the magnitude of the combined acceleration signal, a maximum in the magnitude of the combined acceleration signal, a maximum change versus time of the magnitude of the combined acceleration signal, and a maximum change in the change versus time of the magnitude of the combined acceleration signal. In some cases, the controller 22 may identify the predetermined morphological feature by comparing the magnitude of the combined acceleration signal with a plurality of morphological feature templates, and may identify the predetermined morphological feature as that which corresponds to a matching one of the plurality of morphological feature templates.

The controller 22 is configured to identify a relative time of occurrence of the predetermined morphological feature within the cardiac cycle duration and to determine whether the arrhythmia is an arrhythmia that should be treated by delivery of a therapy by the LCP 10 (and/or treated by another device) or an arrhythmia that should not be treated by the LCP 10 (and/or treated by another device) based at least in part on the relative time of occurrence of the predetermined feature within the cardiac cycle duration.

When the arrhythmia is determined to be an arrhythmia that should be treated by delivery of the therapy by the LCP 10, the controller 22 is configured to deliver the therapy to the heart via two or more electrodes of the LCP. When the arrhythmia is determined to be an arrhythmia that should not be treated by the LCP 10, the controller 22 is configured to not deliver the therapy to the heart via two or more electrodes 14, 16 of the LCP 10. Once the controller 22 determines whether the arrhythmia is an arrhythmia that should be treated by delivery of a therapy by the LCP 10 or an arrhythmia that should not be treated by the LCP 10, the controller 22 may be configured to communicate that determination to one or more other devices such as another implanted LCP or an SICD (Subcutaneous Implantable Cardioverter-Defibrillator). If the controller 22 determines that the arrhythmia is an arrhythmia that should be treated by delivery of a therapy by another device (e.g. by another implanted LCP or an SICD), the controller 22 may be configured to communicate that determination to the other device.

In some cases, the controller 22 may be configured to combine the acceleration signals into the combined acceleration signal by doing one or more of calculating a sum of the acceleration signals from each of the three axes of the three-axis accelerometer 18, calculating a root mean square of the acceleration signals from each of the three axes of the three-axis accelerometer 18, or calculating a root sum square of the acceleration signals from each of the three axes of the three-axis accelerometer 18 into the combined acceleration signal. The combined acceleration signal may have a magnitude that varies with time over each heart cycle duration and may include one or more identifiable morphological features.

In some instances, the controller 22 may be configured to determine whether the arrhythmia is an arrhythmia that should be treated by delivery of the therapy by the LCP 10 (and/or treated by another device) or an arrhythmia that should not be treated by the LCP 10 (and/or treated by another device) based at least in part on whether the relative time of occurrence of the predetermined morphological feature falls within a defined time window of the cardiac cycle duration, wherein the defined time window has a duration that is less than the cardiac cycle duration. The controller 22 may be configured to determine that the arrhythmia is an arrhythmia that should be treated by delivery of the therapy by the LCP 10 (and/or treated by another device) when the relative time of occurrence of the predetermined feature falls within the defined time window of the cardiac cycle duration of the cardiac cycle. In some cases, the defined time window may have a duration that corresponds to a first predetermined percent of the cardiac cycle duration of the particular cardiac cycle and a start time that corresponds to a second predetermined percent of the cardiac cycle duration.

In some cases, the controller 22 may be configured to express a relationship between the cardiac cycle duration and the magnitude of the combined acceleration signal using a polar notation, where the cardiac cycle duration is normalized to 2π radians or 360 degrees, and the relative time of occurrence of the predetermined feature within the cardiac cycle duration is determined by an angular direction at which the predetermined feature is observed in the polar notation. In some cases, a predetermined morphological feature may include one or more of a minimum in the magnitude of the combined acceleration signal, a maximum in the magnitude of the combined acceleration signal, a maximum change versus time of the magnitude of the combined acceleration signal, a maximum change in the change versus time of the magnitude of the combined acceleration signal, and/or some combination or permutation thereof. In some cases, the controller 22 may identify the predetermined morphological feature by comparing the magnitude of the combined acceleration signal with a plurality of morphological feature templates, and may identify the predetermined morphological feature as that which corresponds to a matching one of the plurality of morphological feature templates.

In some instances, the controller 22 may be configured to identify the relative time of occurrence of the predetermined feature within the cardiac cycle duration for each of a plurality of cardiac cycles of the heart, and determine a variability in the relative time of occurrence of the predetermined feature within the cardiac cycle duration among the plurality of cardiac cycles of the heart. In some instances, the variability or stability of the cardiac cycle features are an important indicator of treatable rhythms versus non-treatable rhythms via anti-tachycardia pacing. The controller 22 may be configured to determine whether the arrhythmia is an arrhythmia that should be treated by delivery of a therapy by the LCP 10 (and/or treated by another device) or an arrhythmia that should not be treated by the LCP 10 (and/or treated by another device) based at least in part on the relative time of occurrence of the predetermined feature within the cardiac cycle duration and the variability in the relative time of occurrence of the predetermined feature within the cardiac cycle duration for each of the plurality of cardiac cycles of the heart.

In some instances, the controller 22 may be configured to determine a posture of the patient based at least in part on the acceleration signal of one or more of the three axes of the three-axis accelerometer 18. The controller 22 may be configured to determine whether the arrhythmia is an arrhythmia that should be treated by delivery of a therapy by the LCP 10 (and/or treated by another device) or an arrhythmia that should not be treated by the LCP 10 (and/or treated by another device) based at least in part on the relative time of occurrence of the predetermined feature within the cardiac cycle duration and the posture of the patient. In some instances, posture data may be combined with other cardiac and programming data to optimize the timing of therapy delivery, for example.

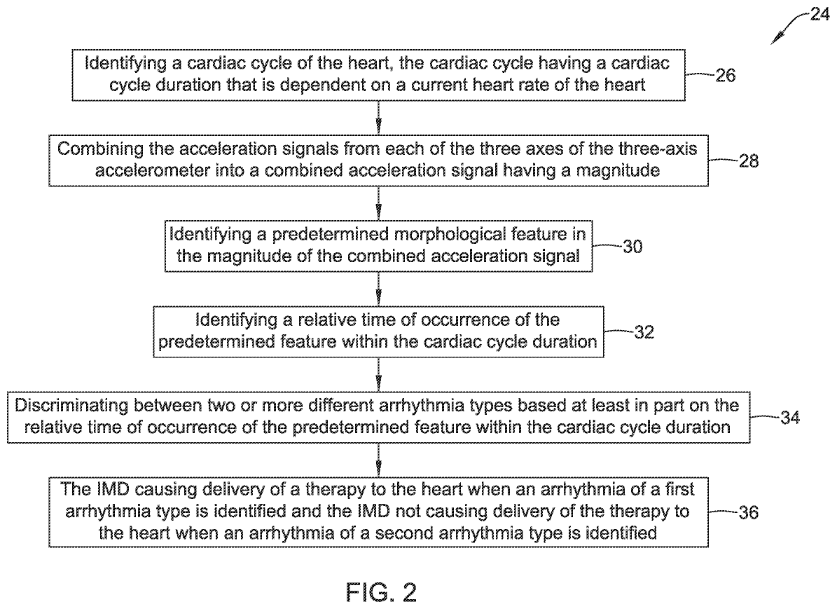

FIG. 2 is a flow diagram showing an illustrative method 24 for operating an implantable medical device (IMD) (such as the LCP 10) for implantation in a heart of a patient, the IMD having a three-axis accelerometer (such as the three-axis accelerometer 18) that provides an acceleration signal for each of the three axes of the three-axis accelerometer. The method 24 includes identifying a cardiac cycle of the heart, the cardiac cycle having a cardiac cycle duration that is dependent on a current heart rate of the heart, as indicated at block 26. The acceleration signals from each of the three axes of the three-axis accelerometer are combined into a combined acceleration signal having a magnitude, as indicated at block 28. A predetermined morphological feature is identified in the magnitude of the combined acceleration signal, as indicated at block 30. A relative time of occurrence of the predetermined feature within the cardiac cycle duration is identified, as indicated at block 32.

The method 24 includes discriminating between two or more different arrhythmia types based at least in part on the relative time of occurrence of the predetermined feature within the cardiac cycle duration, as indicated at block 34. Various types of arrhythmias include, for example, ventricular arrhythmia, supraventricular arrhythmia, paroxysmal supraventricular tachycardia, ventricular tachycardia, monomorphic ventricular tachycardia, polymorphic ventricular tachycardia, ventricular fibrillation, atrial fibrillation, etc. In some cases, discriminating between two or more different arrhythmia types may be based at least in part on whether the relative time of occurrence of the predetermined feature falls within a defined time window of the cardiac cycle duration, wherein the defined time window has a duration that is less than the cardiac cycle duration.

In some cases, the IMD causes delivery of a therapy (by the IMD and/or by another device) to the heart when an arrhythmia of a first arrhythmia type is identified and the IMD does not cause delivery of the therapy (by the IMD and/or by another device) to the heart, or inhibits delivery of the therapy (by the IMD and/or by another device) to the heart, when an arrhythmia of a second arrhythmia type is identified, as indicated at block 36. In some cases, the arrhythmia may be identified as the first arrhythmia type when the relative time of occurrence of the predetermined feature falls within the defined time window of the cardiac cycle duration of the cardiac cycle. The start time and the width of the time window may depend on the cardiac cycle duration of the particular cardiac cycle. That is, in some cases, the start time and the duration of the time window may be normalized to the cardiac cycle duration of the particular cardiac cycle.

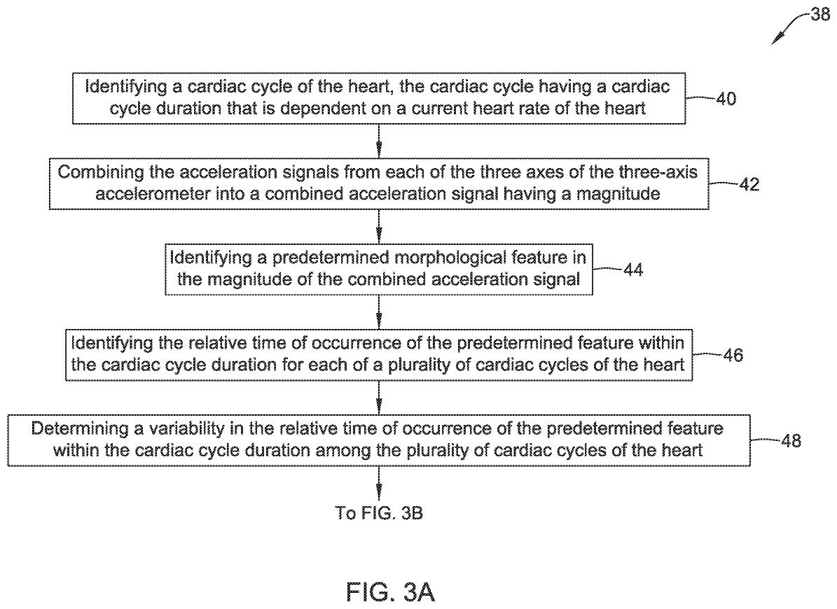

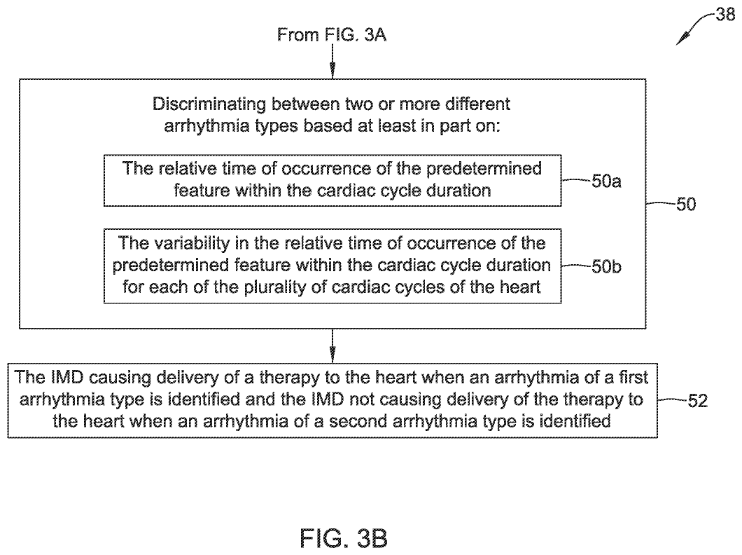

FIGS. 3A and 3B are flow diagrams that together show an illustrative method 38 for operating an implantable medical device (IMD) (such as the LCP 10) for implantation in a heart of a patient. The IMD has a three-axis accelerometer (such as the three-axis accelerometer 18) that provides an acceleration signal for each of the three axes of the three-axis accelerometer. The method 38 includes identifying a cardiac cycle of the heart, the cardiac cycle having a cardiac cycle duration that is dependent on a current heart rate of the heart, as indicated at block 40. The acceleration signals from each of the three axes of the three-axis accelerometer are combined into a combined acceleration signal having a magnitude, as indicated at block 42. By combining the acceleration signals from each of the three axes of the three-axis accelerometer, changes in the orientation of the IMD over time may be factored out from the combined acceleration signal. A predetermined morphological feature is identified in the magnitude of the combined acceleration signal, as indicated at block 30. A relative time of occurrence of the predetermined feature within the cardiac cycle duration is identified, as indicated at block 44.

In some cases, the method 38 includes identifying the relative time of occurrence of the predetermined feature within the cardiac cycle duration for each of a plurality of cardiac cycles of the heart, as indicated at block 46. The method 38 includes determining a variability in the relative time of occurrence of the predetermined feature within the cardiac cycle duration among the plurality of cardiac cycles of the heart, as indicated at block 48.

The method 38 continues with FIG. 3B, with discriminating between two or more different arrhythmia types, as indicated at block 50. Discrimination may be based at least in part on the relative time of occurrence of the predetermined feature within the cardiac cycle duration, as indicated at block 50a. Discrimination may be based at least in part on the variability in the relative time of occurrence of the predetermined feature within the cardiac cycle duration for each of the plurality of cardiac cycles of the heart, as indicated at block 50b. The IMD causes delivery of a therapy (by the IMD and/or by another device) to the heart when an arrhythmia of a first arrhythmia type is identified and the IMD does not cause delivery of the therapy (by the IMD and/or by another device) to the heart, or inhibits delivery of the therapy (by the IMD and/or by another device) to the heart, when an arrhythmia of a second arrhythmia type is identified, as indicated at block 52. In some cases, the arrhythmia may be identified as the first arrhythmia type when the relative time of occurrence of the predetermined feature falls within the defined time window of the cardiac cycle duration of the cardiac cycle.

FIG. 4 is a conceptual schematic block diagram of an illustrative LCP that may be implanted on the heart or within a chamber of the heart and may operate to sense physiological signals and parameters and deliver one or more types of electrical stimulation therapy to the heart of the patient. Example electrical stimulation therapy may include bradycardia pacing, rate responsive pacing therapy, cardiac resynchronization therapy (CRT), anti-tachycardia pacing (ATP) therapy and/or the like. As can be seen in FIG. 4, LCP 100 may be a compact device with all components housed within LCP 100 or directly on housing 120. In some instances, LCP 100 may include communication module 102, pulse generator module 104, electrical sensing module 106, mechanical sensing module 108, processing module 110, energy storage module 112, and electrodes 114. In some cases, the LCP 100 may be considered as being an example of the LCP 10, with the functionality of the controller 22 disposed within the processing module 110, and with the three-axis accelerometer 18 disposed within at least one of the electrical sensing module 106 and the mechanical sensing module 108. In some cases, as will be discussed, the electrical sensing module 106 and the mechanical sensing module 108 may be combined into a single module, and this single module may be or include the three-axis accelerometer 18.

As depicted in FIG. 4, LCP 100 may include electrodes 114, which can be secured relative to housing 120 and electrically exposed to tissue and/or blood surrounding LCP 100. Electrodes 114 may generally conduct electrical signals to and from LCP 100 and the surrounding tissue and/or blood. Such electrical signals can include communication signals, electrical stimulation pulses, and intrinsic cardiac electrical signals, to name a few. Intrinsic cardiac electrical signals may include electrical signals generated by the heart and may be represented by an electrocardiogram (ECG).

Electrodes 114 may include one or more biocompatible conductive materials such as various metals or alloys that are known to be safe for implantation within a human body. In some instances, electrodes 114 may be generally disposed on either end of LCP 100 and may be in electrical communication with one or more of modules 102, 104, 106, 108, and 110. In embodiments where electrodes 114 are secured directly to housing 120, an insulative material may electrically isolate the electrodes 114 from adjacent electrodes, housing 120, and/or other parts of LCP 100. In some instances, some or all of electrodes 114 may be spaced from housing 120 and connected to housing 120 and/or other components of LCP 100 through connecting wires. In such instances, the electrodes 114 may be placed on a tail (not shown) that extends out away from the housing 120. As shown in FIG. 4, in some embodiments, LCP 100 may include electrodes 114′. Electrodes 114′ may be in addition to electrodes 114, or may replace one or more of electrodes 114. Electrodes 114′ may be similar to electrodes 114 except that electrodes 114′ are disposed on the sides of LCP 100. In some cases, electrodes 114′ may increase the number of electrodes by which LCP 100 may deliver communication signals and/or electrical stimulation pulses, and/or may sense intrinsic cardiac electrical signals, communication signals, and/or electrical stimulation pulses.

Electrodes 114 and/or 114′ may assume any of a variety of sizes and/or shapes, and may be spaced at any of a variety of spacings. For example, electrodes 114 may have an outer diameter of two to twenty millimeters (mm). In other embodiments, electrodes 114 and/or 114′ may have a diameter of two, three, five, seven millimeters (mm), or any other suitable diameter, dimension and/or shape. Example lengths for electrodes 114 and/or 114′ may include, for example, one, three, five, ten millimeters (mm), or any other suitable length. As used herein, the length is a dimension of electrodes 114 and/or 114′ that extends away from the outer surface of the housing 120. In some instances, at least some of electrodes 114 and/or 114′ may be spaced from one another by a distance of twenty, thirty, forty, fifty millimeters (mm), or any other suitable spacing. The electrodes 114 and/or 114′ of a single device may have different sizes with respect to each other, and the spacing and/or lengths of the electrodes on the device may or may not be uniform.

In the embodiment shown, communication module 102 may be electrically coupled to electrodes 114 and/or 114′ and may be configured to deliver communication pulses to tissues of the patient for communicating with other devices such as sensors, programmers, other medical devices, and/or the like. Communication signals, as used herein, may be any modulated signal that conveys information to another device, either by itself or in conjunction with one or more other modulated signals. In some embodiments, communication signals may be limited to sub-threshold signals that do not result in capture of the heart yet still convey information. The communication signals may be delivered to another device that is located either external or internal to the patient's body. In some instances, the communication may take the form of distinct communication pulses separated by various amounts of time. In some of these cases, the timing between successive pulses may convey information. Communication module 102 may additionally be configured to sense for communication signals delivered by other devices, which may be located external or internal to the patient's body.

Communication module 102 may communicate to help accomplish one or more desired functions. Some example functions include delivering sensed data, using communicated data for determining occurrences of events such as arrhythmias, coordinating delivery of electrical stimulation therapy, and/or other functions. In some cases, LCP 100 may use communication signals to communicate raw information, processed information, messages and/or commands, and/or other data. Raw information may include information such as sensed electrical signals (e.g. a sensed ECG), signals gathered from coupled sensors, and the like. In some embodiments, the processed information may include signals that have been filtered using one or more signal processing techniques. Processed information may also include parameters and/or events that are determined by the LCP 100 and/or another device, such as a determined heart rate, timing of determined heartbeats, timing of other determined events, determinations of threshold crossings, expirations of monitored time periods, accelerometer signals, activity level parameters, blood-oxygen parameters, blood pressure parameters, heart sound parameters, and the like. Messages and/or commands may include instructions or the like directing another device to take action, notifications of imminent actions of the sending device, requests for reading from the receiving device, requests for writing data to the receiving device, information messages, and/or other messages commands.

In at least some embodiments, communication module 102 (or LCP 100) may further include switching circuitry to selectively connect one or more of electrodes 114 and/or 114′ to communication module 102 in order to select which electrodes 114 and/or 114′ that communication module 102 delivers communication pulses. It is contemplated that communication module 102 may be communicating with other devices via conducted signals, radio frequency (RF) signals, optical signals, acoustic signals, inductive coupling, and/or any other suitable communication methodology. Where communication module 102 generates electrical communication signals, communication module 102 may include one or more capacitor elements and/or other charge storage devices to aid in generating and delivering communication signals. In the embodiment shown, communication module 102 may use energy stored in energy storage module 112 to generate the communication signals. In at least some examples, communication module 102 may include a switching circuit that is connected to energy storage module 112 and, with the switching circuitry, may connect energy storage module 112 to one or more of electrodes 114/114′ to generate the communication signals.

As shown in FIG. 4, a pulse generator module 104 may be electrically connected to one or more of electrodes 114 and/or 114′. Pulse generator module 104 may be configured to generate electrical stimulation pulses and deliver the electrical stimulation pulses to tissues of a patient via one or more of the electrodes 114 and/or 114′ in order to effectuate one or more electrical stimulation therapies. Electrical stimulation pulses as used herein are meant to encompass any electrical signals that may be delivered to tissue of a patient for purposes of treatment of any type of disease or abnormality. For example, when used to treat heart disease, the pulse generator module 104 may generate electrical stimulation pacing pulses for capturing the heart of the patient, i.e. causing the heart to contract in response to the delivered electrical stimulation pulse. In some of these cases, LCP 100 may vary the rate at which pulse generator 104 generates the electrical stimulation pulses, for example in rate adaptive pacing. In other embodiments, the electrical stimulation pulses may include defibrillation/cardioversion pulses for shocking the heart out of fibrillation or into a normal heart rhythm. In yet other embodiments, the electrical stimulation pulses may include anti-tachycardia pacing (ATP) pulses. It should be understood that these are just some examples. When used to treat other ailments, the pulse generator module 104 may generate electrical stimulation pulses suitable for neurostimulation therapy or the like. Pulse generator module 104 may include one or more capacitor elements and/or other charge storage devices to aid in generating and delivering appropriate electrical stimulation pulses. In at least some embodiments, pulse generator module 104 may use energy stored in energy storage module 112 to generate the electrical stimulation pulses. In some particular embodiments, pulse generator module 104 may include a switching circuit that is connected to energy storage module 112 and may connect energy storage module 112 to one or more of electrodes 114/114′ to generate electrical stimulation pulses.

LCP 100 may further include an electrical sensing module 106 and mechanical sensing module 108. Electrical sensing module 106 may be configured to sense intrinsic cardiac electrical signals conducted from electrodes 114 and/or 114′ to electrical sensing module 106. For example, electrical sensing module 106 may be electrically connected to one or more electrodes 114 and/or 114′ and electrical sensing module 106 may be configured to receive cardiac electrical signals conducted through electrodes 114 and/or 114′ via a sensor amplifier or the like. In some embodiments, the cardiac electrical signals may represent local information from the chamber in which LCP 100 is implanted. For instance, if LCP 100 is implanted within a ventricle of the heart, cardiac electrical signals sensed by LCP 100 through electrodes 114 and/or 114′ may represent ventricular cardiac electrical signals. Mechanical sensing module 108 may include, or be electrically connected to, various sensors, such as accelerometers, including multi-axis accelerometers such as two- or three-axis accelerometers, gyroscopes, including multi-axis gyroscopes such as two- or three-axis gyroscopes, blood pressure sensors, heart sound sensors, piezoelectric sensors, blood-oxygen sensors, and/or other sensors which measure one or more physiological parameters of the heart and/or patient. Mechanical sensing module 108, when present, may gather signals from the sensors indicative of the various physiological parameters. Both electrical sensing module 106 and mechanical sensing module 108 may be connected to processing module 110 and may provide signals representative of the sensed cardiac electrical signals and/or physiological signals to processing module 110. Although described with respect to FIG. 4 as separate sensing modules, in some embodiments, electrical sensing module 106 and mechanical sensing module 108 may be combined into a single module. In at least some examples, LCP 100 may only include one of electrical sensing module 106 and mechanical sensing module 108. In some cases, any combination of the processing module 110, electrical sensing module 106, mechanical sensing module 108, communication module 102, pulse generator module 104 and/or energy storage module may be considered a controller of the LCP 100.

Processing module 110 may be configured to direct the operation of LCP 100 and may in some embodiments, be termed a controller. For example, processing module 110 may be configured to receive cardiac electrical signals from electrical sensing module 106 and/or physiological signals from mechanical sensing module 108. Based on the received signals, processing module 110 may determine, for example, occurrences and types of arrhythmias and other determinations such as whether LCP 100 has become dislodged. Processing module 110 may further receive information from communication module 102. In some embodiments, processing module 110 may additionally use such received information to determine occurrences and types of arrhythmias and/or and other determinations such as whether LCP 100 has become dislodged. In still some additional embodiments, LCP 100 may use the received information instead of the signals received from electrical sensing module 106 and/or mechanical sensing module 108—for instance if the received information is deemed to be more accurate than the signals received from electrical sensing module 106 and/or mechanical sensing module 108 or if electrical sensing module 106 and/or mechanical sensing module 108 have been disabled or omitted from LCP 100.

After determining an occurrence of an arrhythmia, processing module 110 may control pulse generator module 104 to generate electrical stimulation pulses in accordance with one or more electrical stimulation therapies to treat the determined arrhythmia. For example, processing module 110 may control pulse generator module 104 to generate pacing pulses with varying parameters and in different sequences to effectuate one or more electrical stimulation therapies. As one example, in controlling pulse generator module 104 to deliver bradycardia pacing therapy, processing module 110 may control pulse generator module 104 to deliver pacing pulses designed to capture the heart of the patient at a regular interval to help prevent the heart of a patient from falling below a predetermined threshold. In some cases, the rate of pacing may be increased with an increased activity level of the patient (e.g. rate adaptive pacing). For instance, processing module 110 may monitor one or more physiological parameters of the patient which may indicate a need for an increased heart rate (e.g. due to increased metabolic demand). Processing module 110 may then increase the rate at which pulse generator 104 generates electrical stimulation pulses.

For ATP therapy, processing module 110 may control pulse generator module 104 to deliver pacing pulses at a rate faster than an intrinsic heart rate of a patient in attempt to force the heart to beat in response to the delivered pacing pulses rather than in response to intrinsic cardiac electrical signals. Once the heart is following the pacing pulses, processing module 110 may control pulse generator module 104 to reduce the rate of delivered pacing pulses down to a safer level. In CRT, processing module 110 may control pulse generator module 104 to deliver pacing pulses in coordination with another device to cause the heart to contract more efficiently. In cases where pulse generator module 104 is capable of generating defibrillation and/or cardioversion pulses for defibrillation/cardioversion therapy, processing module 110 may control pulse generator module 104 to generate such defibrillation and/or cardioversion pulses. In some cases, processing module 110 may control pulse generator module 104 to generate electrical stimulation pulses to provide electrical stimulation therapies different than those examples described above.

Aside from controlling pulse generator module 104 to generate different types of electrical stimulation pulses and in different sequences, in some embodiments, processing module 110 may also control pulse generator module 104 to generate the various electrical stimulation pulses with varying pulse parameters. For example, each electrical stimulation pulse may have a pulse width and a pulse amplitude. Processing module 110 may control pulse generator module 104 to generate the various electrical stimulation pulses with specific pulse widths and pulse amplitudes. For example, processing module 110 may cause pulse generator module 104 to adjust the pulse width and/or the pulse amplitude of electrical stimulation pulses if the electrical stimulation pulses are not effectively capturing the heart. Such control of the specific parameters of the various electrical stimulation pulses may help LCP 100 provide more effective delivery of electrical stimulation therapy.

In some embodiments, processing module 110 may further control communication module 102 to send information to other devices. For example, processing module 110 may control communication module 102 to generate one or more communication signals for communicating with other devices of a system of devices. For instance, processing module 110 may control communication module 102 to generate communication signals in particular pulse sequences, where the specific sequences convey different information. Communication module 102 may also receive communication signals for potential action by processing module 110.

In further embodiments, processing module 110 may control switching circuitry by which communication module 102 and pulse generator module 104 deliver communication signals and/or electrical stimulation pulses to tissue of the patient. As described above, both communication module 102 and pulse generator module 104 may include circuitry for connecting one or more electrodes 114 and/114′ to communication module 102 and/or pulse generator module 104 so those modules may deliver the communication signals and electrical stimulation pulses to tissue of the patient. The specific combination of one or more electrodes by which communication module 102 and/or pulse generator module 104 deliver communication signals and electrical stimulation pulses may influence the reception of communication signals and/or the effectiveness of electrical stimulation pulses. Although it was described that each of communication module 102 and pulse generator module 104 may include switching circuitry, in some embodiments, LCP 100 may have a single switching module connected to the communication module 102, the pulse generator module 104, and electrodes 114 and/or 114′. In such embodiments, processing module 110 may control the switching module to connect modules 102/104 and electrodes 114/114′ as appropriate.

In some embodiments, processing module 110 may include a pre-programmed chip, such as a very-large-scale integration (VLSI) chip or an application specific integrated circuit (ASIC). In such embodiments, the chip may be pre-programmed with control logic in order to control the operation of LCP 100. By using a pre-programmed chip, processing module 110 may use less power than other programmable circuits while able to maintain basic functionality, thereby potentially increasing the battery life of LCP 100. In other instances, processing module 110 may include a programmable microprocessor or the like. Such a programmable microprocessor may allow a user to adjust the control logic of LCP 100 after manufacture, thereby allowing for greater flexibility of LCP 100 than when using a pre-programmed chip. In still other embodiments, processing module 110 may not be a single component. For example, processing module 110 may include multiple components positioned at disparate locations within LCP 100 in order to perform the various described functions. For example, certain functions may be performed in one component of processing module 110, while other functions are performed in a separate component of processing module 110.

Processing module 110, in additional embodiments, may include a memory circuit and processing module 110 may store information on and read information from the memory circuit. In other embodiments, LCP 100 may include a separate memory circuit (not shown) that is in communication with processing module 110, such that processing module 110 may read and write information to and from the separate memory circuit. The memory circuit, whether part of processing module 110 or separate from processing module 110, may be volatile memory, non-volatile memory, or a combination of volatile memory and non-volatile memory.

Energy storage module 112 may provide a power source to LCP 100 for its operations. In some embodiments, energy storage module 112 may be a non-rechargeable lithium-based battery. In other embodiments, the non-rechargeable battery may be made from other suitable materials. In some embodiments, energy storage module 112 may include a rechargeable battery. In still other embodiments, energy storage module 112 may include other types of energy storage devices such as capacitors or super capacitors.

To implant LCP 100 inside a patient's body, an operator (e.g., a physician, clinician, etc.), may fix LCP 100 to the cardiac tissue of the patient's heart. To facilitate fixation, LCP 100 may include one or more anchors 116. The one or more anchors 116 are shown schematically in FIG. 4. The one or more anchors 116 may include any number of fixation or anchoring mechanisms. For example, one or more anchors 116 may include one or more pins, staples, threads, screws, helix, tines, and/or the like. In some embodiments, although not shown, one or more anchors 116 may include threads on its external surface that may run along at least a partial length of an anchor member. The threads may provide friction between the cardiac tissue and the anchor to help fix the anchor member within the cardiac tissue. In some cases, the one or more anchors 116 may include an anchor member that has a cork-screw shape that can be screwed into the cardiac tissue. In other embodiments, anchor 116 may include other structures such as barbs, spikes, or the like to facilitate engagement with the surrounding cardiac tissue.

In some examples, LCP 100 may be configured to be implanted on a patient's heart or within a chamber of the patient's heart. For instance, LCP 100 may be implanted within any of a left atrium, right atrium, left ventricle, or right ventricle of a patient's heart. By being implanted within a specific chamber, LCP 100 may be able to sense cardiac electrical signals originating or emanating from the specific chamber that other devices may not be able to sense with such resolution. Where LCP 100 is configured to be implanted on a patient's heart, LCP 100 may be configured to be implanted on or adjacent to one of the chambers of the heart, or on or adjacent to a path along which intrinsically generated cardiac electrical signals generally follow. In these examples, LCP 100 may also have an enhanced ability to sense localized intrinsic cardiac electrical signals and deliver localized electrical stimulation therapy.

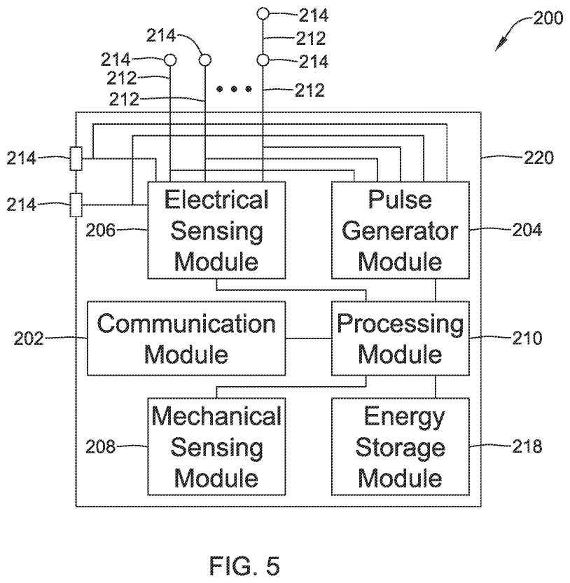

FIG. 5 depicts an embodiment of another device, medical device (MD) 200, which may operate to sense physiological signals and parameters and deliver one or more types of electrical stimulation therapy to tissues of the patient. The MD 200 may be considered as being an example of another device to which the LCP 10 may communicate its decision as to whether or not to provide therapy. In some cases, this communication may also include instructions for the MD 200 to provide therapy or, alternatively, to withhold or inhibit therapy, depending on the nature of the detected arrhythmia.

In the embodiment shown, MD 200 may include a communication module 202, a pulse generator module 204, an electrical sensing module 206, a mechanical sensing module 208, a processing module 210, and an energy storage module 218. Each of modules 202, 204, 206, 208, and 210 may be similar to modules 102, 104, 106, 108, and 110 of LCP 100. Additionally, energy storage module 218 may be similar to energy storage module 112 of LCP 100. However, in some embodiments, MD 200 may have a larger volume within housing 220. In such embodiments, MD 200 may include a larger energy storage module 218 and/or a larger processing module 210 capable of handling more complex operations than processing module 110 of LCP 100.

While MD 200 may be another leadless device such as shown in FIG. 4, in some instances MD 200 may include leads, such as leads 212. Leads 212 may include electrical wires that conduct electrical signals between electrodes 214 and one or more modules located within housing 220. In some cases, leads 212 may be connected to and extend away from housing 220 of MD 200. In some embodiments, leads 212 are implanted on, within, or adjacent to a heart of a patient. Leads 212 may contain one or more electrodes 214 positioned at various locations on leads 212 and various distances from housing 220. Some leads 212 may only include a single electrode 214, while other leads 212 may include multiple electrodes 214. Generally, electrodes 214 are positioned on leads 212 such that when leads 212 are implanted within the patient, one or more of the electrodes 214 are positioned to perform a desired function. In some cases, the one or more of the electrodes 214 may be in contact with the patient's cardiac tissue. In other cases, the one or more of the electrodes 214 may be positioned subcutaneously but adjacent the patient's heart. The electrodes 214 may conduct intrinsically generated electrical cardiac signals to leads 212. Leads 212 may in turn, conduct the received electrical cardiac signals to one or more of the modules 202, 204, 206, and 208 of MD 200. In some cases, MD 200 may generate electrical stimulation signals, and leads 212 may conduct the generated electrical stimulation signals to electrodes 214. Electrodes 214 may then conduct the electrical stimulation signals to the cardiac tissue of the patient (either directly or indirectly). MD 200 may also include one or more electrodes 214 not disposed on a lead 212. For example, one or more electrodes 214 may be connected directly to housing 220.

Leads 212, in some embodiments, may additionally contain one or more sensors, such as accelerometers, blood pressure sensors, heart sound sensors, blood-oxygen sensors, and/or other sensors which are configured to measure one or more physiological parameters of the heart and/or patient. In such embodiments, mechanical sensing module 208 may be in electrical communication with leads 212 and may receive signals generated from such sensors.

While not required, in some embodiments MD 200 may be an implantable medical device. In such embodiments, housing 220 of MD 200 may be implanted in, for example, a transthoracic region of the patient. Housing 220 may generally include any of a number of known materials that are safe for implantation in a human body and may when implanted, hermetically seal the various components of MD 200 from fluids and tissues of the patient's body. In such embodiments, leads 212 may be implanted at one or more various locations within the patient, such as within the heart of the patient, adjacent to the heart of the patient, adjacent to the spine of the patient, or any other desired location.

In some embodiments, MD 200 may be an implantable cardiac pacemaker (ICP). In these embodiments, MD 200 may have one or more leads, for example leads 212, which are implanted on or within the patient's heart. The one or more leads 212 may include one or more electrodes 214 that are in contact with cardiac tissue and/or blood of the patient's heart. MD 200 may be configured to sense intrinsically generated cardiac electrical signals and determine, for example, one or more cardiac arrhythmias based on analysis of the sensed signals. MD 200 may be configured to deliver CRT, ATP therapy, bradycardia therapy, and/or other therapy types via leads 212 implanted within the heart. In some embodiments, MD 200 may additionally be configured to provide defibrillation/cardioversion therapy.

In some instances, MD 200 may be an implantable cardioverter-defibrillator (ICD). In such embodiments, MD 200 may include one or more leads implanted within a patient's heart. MD 200 may also be configured to sense electrical cardiac signals, determine occurrences of tachyarrhythmias based on the sensed electrical cardiac signals, and deliver defibrillation and/or cardioversion therapy in response to determining an occurrence of a tachyarrhythmia (for example by delivering defibrillation and/or cardioversion pulses to the heart of the patient). In other embodiments, MD 200 may be a subcutaneous implantable cardioverter-defibrillator (SICD). In embodiments where MD 200 is an SICD, one of leads 212 may be a subcutaneously implanted lead. In at least some embodiments where MD 200 is an SICD, MD 200 may include only a single lead which is implanted subcutaneously but outside of the chest cavity, however this is not required.

In some embodiments, MD 200 may not be an implantable medical device. Rather, MD 200 may be a device external to the patient's body, and electrodes 214 may be skin-electrodes that are placed on a patient's body. In such embodiments, MD 200 may be able to sense surface electrical signals (e.g. electrical cardiac signals that are generated by the heart or electrical signals generated by a device implanted within a patient's body and conducted through the body to the skin). MD 200 may further be configured to deliver various types of electrical stimulation therapy, including, for example, defibrillation therapy via skin-electrodes 214.

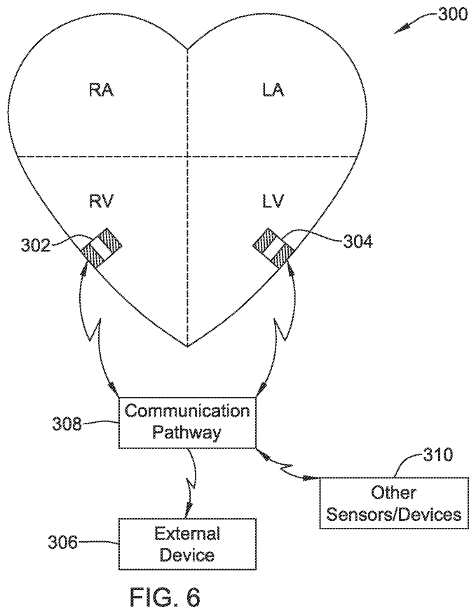

FIG. 6 illustrates an embodiment of a medical device system and a communication pathway through which multiple medical devices 302, 304, 306, and/or 310 of the medical device system may communicate. In the embodiment shown, medical device system 300 may include LCPs 302 and 304, external medical device 306, and other sensors/devices 310. External device 306 may be a device disposed external to a patient's body, as described previously with respect to MD 200. In at least some examples, external device 306 may represent an external support device such as a device programmer, as will be described in more detail below. Other sensors/devices 310 may be any of the devices described previously with respect to MD 200, such as ICPs, ICDs, and SICDs. Other sensors/devices 310 may also include various diagnostic sensors that gather information about the patient, such as accelerometers, blood pressure sensors, or the like. In some cases, other sensors/devices 310 may include an external programmer device that may be used to program one or more devices of system 300.

One of the LCP 302 and the LCP 304 may be considered as representing the LCP 10, and the other of the LCP 302 and the LCP 304 may be considered as being as being an example of another device to which the LCP 10 may communicate its decision as to whether or not to provide therapy. In some cases, this communication may also include instructions for the other of the LCP 302 and the LCP 304 to provide therapy or, alternatively, to withhold therapy, depending on the nature of the detected arrhythmia.

Various devices of system 300 may communicate via communication pathway 308. For example, LCPs 302 and/or 304 may sense intrinsic cardiac electrical signals and may communicate such signals to one or more other devices 302/304, 306, and 310 of system 300 via communication pathway 308. In one embodiment, one or more of devices 302/304 may receive such signals and, based on the received signals, determine an occurrence of an arrhythmia. In some cases, device or devices 302/304 may communicate such determinations to one or more other devices 306 and 310 of system 300. In some cases, one or more of devices 302/304, 306, and 310 of system 300 may take action based on the communicated determination of an arrhythmia, such as by delivering a suitable electrical stimulation to the heart of the patient. One or more of devices 302/304, 306, and 310 of system 300 may additionally communicate command or response messages via communication pathway 308. The command messages may cause a receiving device to take a particular action whereas response messages may include requested information or a confirmation that a receiving device did, in fact, receive a communicated message or data.

It is contemplated that the various devices of system 300 may communicate via pathway 308 using RF signals, inductive coupling, optical signals, acoustic signals, or any other signals suitable for communication. Additionally, in at least some embodiments, the various devices of system 300 may communicate via pathway 308 using multiple signal types. For instance, other sensors/device 310 may communicate with external device 306 using a first signal type (e.g. RF communication) but communicate with LCPs 302/304 using a second signal type (e.g. conducted communication). Further, in some embodiments, communication between devices may be limited. For instance, as described above, in some embodiments, LCPs 302/304 may communicate with external device 306 only through other sensors/devices 310, where LCPs 302/304 send signals to other sensors/devices 310, and other sensors/devices 310 relay the received signals to external device 306.

In some cases, the various devices of system 300 may communicate via pathway 308 using conducted communication signals. Accordingly, devices of system 300 may have components that allow for such conducted communication. For instance, the devices of system 300 may be configured to transmit conducted communication signals (e.g. a voltage and/or current waveform punctuated with current and/or voltage pulses, referred herein as electrical communication pulses) into the patient's body via one or more electrodes of a transmitting device, and may receive the conducted communication signals via one or more electrodes of a receiving device. The patient's body may “conduct” the conducted communication signals from the one or more electrodes of the transmitting device to the electrodes of the receiving device in the system 300. In such embodiments, the delivered conducted communication signals may differ from pacing pulses, defibrillation and/or cardioversion pulses, or other electrical stimulation therapy signals. For example, the devices of system 300 may deliver electrical communication pulses at an amplitude/pulse width that is sub-threshold. That is, the communication pulses have an amplitude/pulse width designed to not capture the heart. In some cases, the amplitude/pulse width of the delivered electrical communication pulses may be above the capture threshold of the heart, but may be delivered during a refractory period of the heart and/or may be incorporated in or modulated onto a pacing pulse, if desired.

Additionally, unlike normal electrical stimulation therapy pulses, the electrical communication pulses may be delivered in specific sequences which convey information to receiving devices. For instance, delivered electrical communication pulses may be modulated in any suitable manner to encode communicated information. In some cases, the communication pulses may be pulse width modulated and/or amplitude modulated. Alternatively, or in addition, the time between pulses may be modulated to encode desired information. In some cases, a predefined sequence of communication pulses may represent a corresponding symbol (e.g. a logic “1” symbol, a logic “0” symbol, an ATP therapy trigger symbol, etc.). In some cases, conducted communication pulses may be voltage pulses, current pulses, biphasic voltage pulses, biphasic current pulses, or any other suitable electrical pulse as desired.

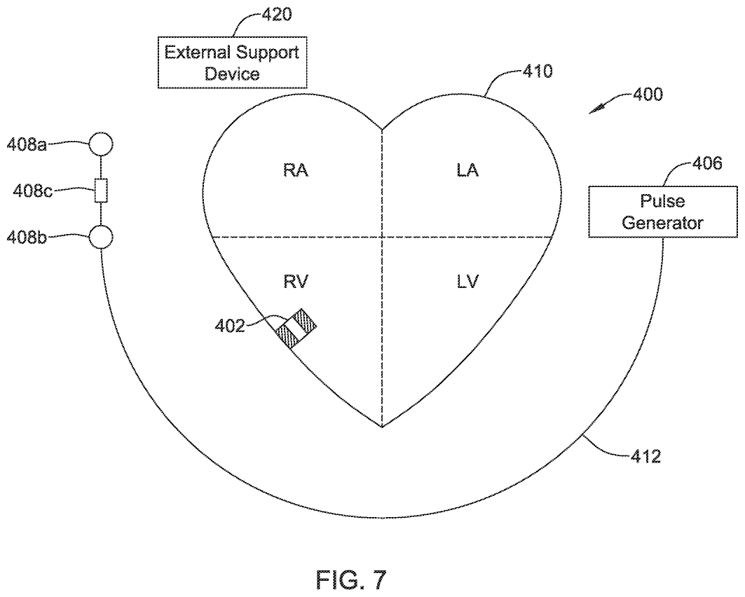

FIG. 7 depicts an illustrative medical device system 400 that may be configured to operate together. For example, system 400 may include multiple devices that are implanted within a patient and are configured to sense physiological signals, determine occurrences of cardiac arrhythmias, and deliver electrical stimulation to treat detected cardiac arrhythmias. In some embodiments, the devices of system 400 may be configured to determine occurrences of dislodgment of one or more devices of system 400. In FIG. 7, an LCP 402 is shown fixed to the interior of the right ventricle of the heart 410, and a pulse generator 406 is shown coupled to a lead 412 having one or more electrodes 408a-408c. In some cases, pulse generator 406 may be part of a subcutaneous implantable cardioverter-defibrillator (SICD), and the one or more electrodes 408a-408c may be positioned subcutaneously adjacent the heart. LCP 402 may communicate with the SICD, such as via communication pathway 308. The locations of LCP 402, pulse generator 406, lead 412, and electrodes 408a-c depicted in FIG. 7 are just exemplary. In other embodiments of system 400, LCP 402 may be positioned in the left ventricle, right atrium, or left atrium of the heart, as desired. In still other embodiments, LCP 402 may be implanted externally adjacent to heart 410 or even remote from heart 410.

The LCP 402 may represent the LCP 10. The pulse generator 406, which as noted may be part of an SICD, may be considered as being an example of another device to which the LCP 10 may communicate its decision as to whether or not to provide therapy. In some cases, this communication may also include instructions for the pulse generator 406 to provide therapy, including delivering a shock or, alternatively, to withhold therapy, depending on the nature of the detected arrhythmia.

Medical device system 400 may also include external support device 420. External support device 420 can be used to perform functions such as device identification, device programming and/or transfer of real-time and/or stored data between devices using one or more of the communication techniques described herein, or other functions involving communication with one or more devices of system 400. As one example, communication between external support device 420 and pulse generator 406 can be performed via a wireless mode, and communication between pulse generator 406 and LCP 402 can be performed via a conducted communication mode. In some embodiments, communication between LCP 402 and external support device 420 is accomplished by sending communication information through pulse generator 406. However, in other embodiments, communication between the LCP 402 and external support device 420 may be via a communication module.

FIG. 7 only illustrates one example embodiment of a medical device system that may be configured to operate according to techniques disclosed herein. Other example medical device systems may include additional or different medical devices and/or configurations. For instance, other medical device systems that are suitable to operate according to techniques disclosed herein may include additional LCPs implanted within the heart. Another example medical device system may include a plurality of LCPs with or without other devices such as pulse generator 406, with at least one LCP capable of delivering defibrillation therapy. Still another example may include one or more LCPs implanted along with a transvenous pacemaker and with or without an implanted SICD. In yet other embodiments, the configuration or placement of the medical devices, leads, and/or electrodes may be different from those depicted in FIG. 4. Accordingly, it should be recognized that numerous other medical device systems, different from system 400 depicted in FIG. 7, may be operated in accordance with techniques disclosed herein. As such, the embodiment shown in FIG. 7 should not be viewed as limiting in any way.

In some embodiments, LCP 100 may be configured to operate in one or more modes. Within each mode, LCP 100 may operate in a somewhat different manner. For instance, in a first mode, LCP 100 may be configured to sense certain signals and/or determine certain parameters. In a second mode, LCP 100 may be configured to sense the signals differently, sense at least some different signals, and/or determine at least some different parameters than in the first mode. In at least one mode, LCP 100 may be configured to confirm whether an arrhythmia of a patient's heart is occurring. For ease of description, a mode that includes LCP 100 being configured to confirm whether an arrhythmia of a patient's heart is occurring may be called an arrhythmia confirmation mode. Other modes may include one or more programming and/or therapy modes, and it may be possible for LCP 100 to be engaged in multiple modes concurrently.

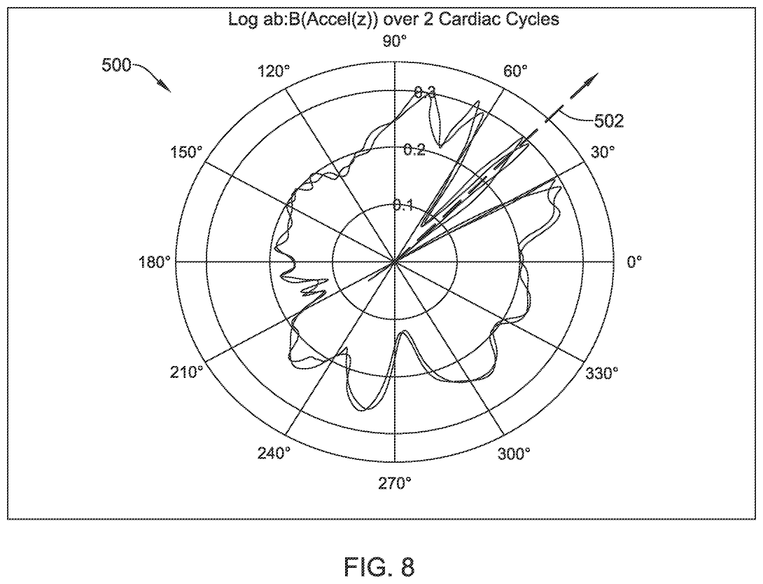

FIGS. 8 through 10 are polar graphs of the magnitude of a combined acceleration signal sensed by a three-axis accelerometer of an LCP implanted in the ventricle of a canine's heart. While each cardiac cycle may have a different cardiac cycle duration, the magnitude of the combined acceleration signal is normalized in time to 2π radians (or 360 degrees). It will be appreciated that each graph includes a notch, which represents a minimum acceleration magnitude. In this example, the notch represents a predetermined morphological feature in the magnitude of the combined acceleration signal. This notch may be identified by, for example, one or more of a minimum in the magnitude of the combined acceleration signal, a maximum change versus time of the magnitude of the combined acceleration signal, and a maximum change in the change versus time of the magnitude of the combined acceleration signal. Alternatively, or in addition, this notch may be identified by comparing the magnitude of the combined acceleration signal with a plurality of morphological feature templates, and may identify the notch as that which corresponds to a matching one of the plurality of morphological feature templates. These are just examples.

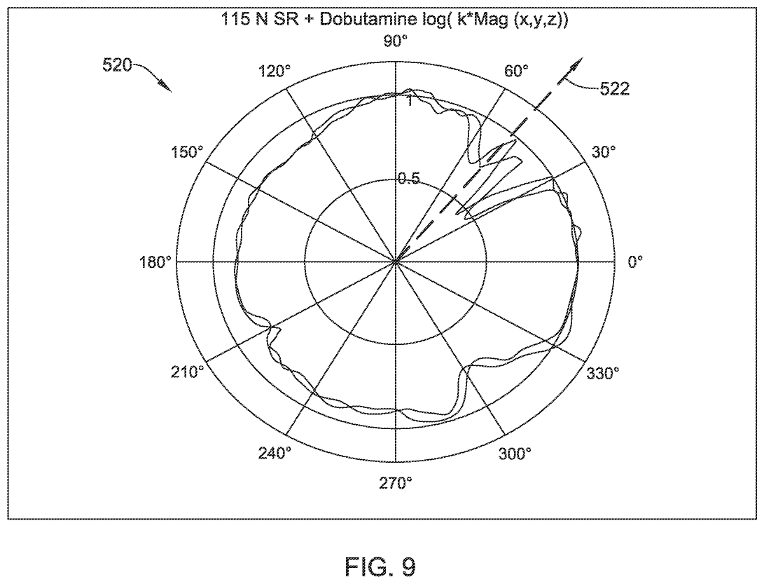

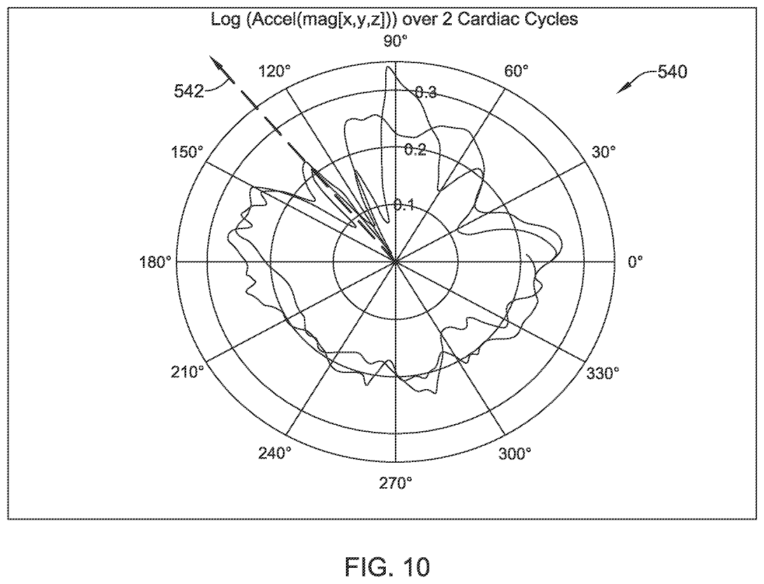

The relative angle that this notch has during the cardiac cycle (e.g. phase angle) can be interpreted as being an indication of the type of cardiac rhythm or arrhythmia. The relative angle in polar notation represents the relative time of occurrence of the predetermined morphological feature (e.g. notch) within the cardiac cycle duration (here normalized to 360 degrees). The data used to form these graphs is canine data, and was sampled at 200 Hz. FIG. 8 includes a plot 500 that represents the magnitude of the combined acceleration signal for a normal sinus rhythm. The plot 500 can be seen as including a notch representing a phase angle of about 40 degrees, as indicated by an arrow 502. In one example, the normal sinus rhythm may be defined at least in part by a phase angle that falls within a time window represented by a phase angle range of 35-45 degrees. FIG. 9 includes a plot 520 which represents the magnitude of the combined acceleration signal for a rhythm under the influence of dobutamine, representing an arrhythmia originating in the atrium (e.g. atrial fibrillation). The plot 520 can be seen as including a notch representing a phase angle of about 50 or 55 degrees, as indicated by an arrow 522. In one example, an atrial fibrillation may be defined at least in part by a phase angle that falls within a time window represented by a phase angle range of 45-60 degrees. FIG. 10 includes a plot 540 which represents the magnitude of the combined acceleration signal for a paced rhythm. The plot 540 can be seen as including a notch representing a phase angle of about 130 degrees, as indicated by an arrow 542. In one example, a paced rhythm may be defined at least in part by a phase angle that falls within a time window represented by a phase angle range of 120-140 degrees. It can be seen that the plot 540, representing a paced rhythm, is significantly different from either the plot 500 or the plot 520. These are just examples.

Rather than using a polar notation, it is contemplated that the magnitude of the combined acceleration signal may be expressed in a rectangular notation with time along the “X” axis and magnitude along the “Y” axis. In rectangular notation, each cardiac cycle may be identified along the “X” axis, and the corresponding cardiac cycle duration may be determined. The cardiac cycle duration will be dependent on the current heart rate of the heart. For each cardiac cycle, a predetermined morphological feature may be identified in the magnitude of the combined acceleration signal. A relative time of occurrence of the predetermined morphological feature within the corresponding cardiac cycle duration may be used to determine whether an arrhythmia is an arrhythmia that should be treated by delivery of a therapy to the heart or an arrhythmia that should not be treated.

In some cases, a time window is defined along the “X” axis for each cardiac cycle, where the time window has a duration that is less than the corresponding cardiac cycle duration. In some cases, each time window may have a duration that corresponds to a predetermined percent of the corresponding cardiac cycle duration and a start time that corresponds to a predetermined percent of the corresponding cardiac cycle duration. For example, a time window may be defined as having a duration of 5 percent of the corresponding cardiac cycle duration, and a start time that corresponds to 33 percent of the corresponding cardiac cycle duration. Such time windows expressed in the rectangular notation may correspond to the phase angle ranges discussed with respect to the polar notation.

Although various features may have been described with respect to less than all embodiments, this disclosure contemplates that those features may be included on any embodiment. Further, although the embodiments described herein may have omitted some combinations of the various described features, this disclosure contemplates embodiments that include any combination of each described feature. Accordingly, departure in form and detail may be made without departing from the scope and spirit of the present disclosure as described in the appended claims.

What is claimed is:

1. A leadless cardiac pacemaker (LCP) for implantation in a heart of a patient, the LCP comprising:
a housing;
two or more electrodes exposed to an exterior of the housing;
a three-axis accelerometer disposed within the housing, the three-axis accelerometer providing an acceleration signal for each of the three axes of the three-axis accelerometer;
a memory;
a controller disposed within the housing and operably coupled with the two or more electrodes, the three-axis accelerometer and the memory, the controller configured to:
identify a cardiac cycle of the heart, the cardiac cycle having a cardiac cycle duration that is dependent on a current heart rate of the heart;
determine the heart is experiencing an arrhythmia;
receive the acceleration signal from each of the three axes of the three-axis accelerometer;
combine the acceleration signals from each of the three axes of the three-axis accelerometer into a combined acceleration signal having a magnitude;
identify a predetermined morphological feature in the magnitude of the combined acceleration signal;
identify a relative time of occurrence of the predetermined feature within the cardiac cycle duration;
determine whether the arrhythmia is an arrhythmia that should be treated by delivery of a therapy by the LCP or an arrhythmia that should not be treated by the LCP based at least in part on the relative time of occurrence of the predetermined feature within the cardiac cycle duration;
when the arrhythmia is determined to be an arrhythmia that should be treated by delivery of the therapy by the LCP, the controller configured to deliver the therapy to the heart via two or more electrodes of the LCP; and
when the arrhythmia is determined to be an arrhythmia that should not be treated by the LCP, the controller is configured to not deliver the therapy to the heart via two or more electrodes of the LCP.

2. The LCP of claim 1, wherein the controller is configured to combine the acceleration signals into the combined acceleration signal by:
calculating a sum of the acceleration signals from each of the three axes of the three-axis accelerometer;
calculating a root mean square of the acceleration signals from each of the three axes of the three-axis accelerometer; or
calculating a root sum square of the acceleration signals from each of the three axes of the three-axis accelerometer into the combined acceleration signal.

3. The LCP of claim 1, wherein the controller is configured to:
receive an electrocardiogram signal via the two or more electrodes that are exposed to the exterior of the housing; and
identify the cardiac cycle of the heart based at least in part on the electrocardiogram signal.

4. The LCP of claim 3, wherein the controller is configured to determine the heart is experiencing an arrhythmia based at least in part on the electrocardiogram signal.

5. The LCP of claim 1, wherein the controller is configured to determine the heart is experiencing an arrhythmia based at least in part on the acceleration signal of one or more of the three axes of the three-axis accelerometer.

6. The LCP of claim 1, wherein the controller is configured to determine whether the arrhythmia is an arrhythmia that should be treated by delivery of the therapy by the LCP or an arrhythmia that should not be treated by the LCP based at least in part on whether the relative time of occurrence of the predetermined feature falls within a defined time window of the cardiac cycle duration, wherein the defined time window has a duration that is less than the cardiac cycle duration.

7. The LCP of claim 6, wherein the controller is configured to determine that the arrhythmia is an arrhythmia that should be treated by delivery of the therapy by the LCP when the relative time of occurrence of the predetermined feature falls within the defined time window of the cardiac cycle duration of the cardiac cycle.

8. The LCP of claim 7, wherein the defined time window has a duration that corresponds to a first predetermined percent of the cardiac cycle duration and a start time that corresponds to a second predetermined percent of the cardiac cycle duration.

9. The LCP of claim 1, wherein once the controller determines whether the arrhythmia is an arrhythmia that should be treated by delivery of a therapy by the LCP or an arrhythmia that should not be treated by the LCP, the controller is configured to communicate that determination to one or more other devices.

10. The LCP of claim 1, wherein the controller is configured to express a relationship between the cardiac cycle duration and the magnitude of the combined acceleration signal using a polar notation, where the cardiac cycle duration is normalized to 2π radians or 360 degrees, and the relative time of occurrence of the predetermined feature within the cardiac cycle duration is determined by an angular direction that the predetermined feature is observed in the polar notation.

11. The LCP of claim 1, wherein the predetermined morphological feature comprises one or more of:
a minimum in the magnitude of the combined acceleration signal;
a maximum in the magnitude of the combined acceleration signal;
a maximum change versus time of the magnitude of the combined acceleration signal; and
a maximum change in the change versus time of the magnitude of the combined acceleration signal.

12. The LCP of claim 1, wherein the controller identifies the predetermined morphological feature by comparing the magnitude of the combined acceleration signal with a plurality of morphological feature templates, and identifies the predetermined morphological feature as that which corresponds to a matching one of the plurality of morphological feature templates.

13. The LCP of claim 1, wherein the controller is configured to:
identify the relative time of occurrence of the predetermined feature within the cardiac cycle duration for each of a plurality of cardiac cycles of the heart;
determine a variability in the relative time of occurrence of the predetermined feature within the cardiac cycle duration among the plurality of cardiac cycles of the heart;
determine whether the arrhythmia is an arrhythmia that should be treated by delivery of a therapy by the LCP or an arrhythmia that should not be treated by the LCP based at least in part on:
the relative time of occurrence of the predetermined feature within the cardiac cycle duration; and
the variability in the relative time of occurrence of the predetermined feature within the cardiac cycle duration for each of the plurality of cardiac cycles of the heart.

14. The LCP of claim 1, wherein the controller is further configured to:
determine a posture of the patient based at least in part on the acceleration signal of one or more of the three axes of the three-axis accelerometer;
determine whether the arrhythmia is an arrhythmia that should be treated by delivery of a therapy by the LCP or an arrhythmia that should not be treated by the LCP based at least in part on:
the relative time of occurrence of the predetermined feature within the cardiac cycle duration; and
the posture of the patient.

15. A method for operating an implantable medical device (IMD) for implantation in a heart of a patient, the IMD having a three-axis accelerometer that provides an acceleration signal for each of the three axes of the three-axis accelerometer, the method comprising:
identifying a cardiac cycle of the heart, the cardiac cycle having a cardiac cycle duration that is dependent on a current heart rate of the heart;
combining the acceleration signals from each of the three axes of the three-axis accelerometer into a combined acceleration signal having a magnitude;
identifying a predetermined morphological feature in the magnitude of the combined acceleration signal;
identifying a relative time of occurrence of the predetermined feature within the cardiac cycle duration;
discriminating between two or more different arrhythmia types based at least in part on the relative time of occurrence of the predetermined feature within the cardiac cycle duration; and
the IMD causing delivery of a therapy to the heart when an arrhythmia of a first arrhythmia type is identified and the IMD not causing delivery of the therapy to the heart when an arrhythmia of a second arrhythmia type is identified.

16. The method of claim 15, wherein discriminating between two or more different arrhythmia types is based at least in part on whether the relative time of occurrence of the predetermined feature falls within a defined time window of the cardiac cycle duration, wherein the defined time window has a duration that is less than the cardiac cycle duration.

17. The method of 16, wherein the arrhythmia is identified as the first arrhythmia type when the relative time of occurrence of the predetermined feature falls within the defined time window of the cardiac cycle duration of the cardiac cycle.

18. The method of 15, comprising:
identifying the relative time of occurrence of the predetermined feature within the cardiac cycle duration for each of a plurality of cardiac cycles of the heart;
determining a variability in the relative time of occurrence of the predetermined feature within the cardiac cycle duration among the plurality of cardiac cycles of the heart;
discriminating between two or more different arrhythmia types based at least in part on:
the relative time of occurrence of the predetermined feature within the cardiac cycle duration; and
the variability in the relative time of occurrence of the predetermined feature within the cardiac cycle duration for each of the plurality of cardiac cycles of the heart.

19. A non-transitory computer readable medium storing instructions that when executed by one or more processors cause the one or more processors to:
identify a cardiac cycle of a heart, the cardiac cycle having a cardiac cycle duration that is dependent on a current heart rate of the heart;
combine acceleration signals from each of three axes of a three-axis accelerometer into a combined acceleration signal having a magnitude;
identify a predetermined morphological feature in the magnitude of the combined acceleration signal;
identify a relative time of occurrence of the predetermined feature within the cardiac cycle duration;
discriminate between two or more different arrhythmia types based at least in part on the relative time of occurrence of the predetermined feature within the cardiac cycle duration; and
cause delivery of a therapy to the heart when an arrhythmia of a first arrhythmia type is identified and not causing delivery of the therapy to the heart when an arrhythmia of a second arrhythmia type is identified.

20. The non-transitory computer readable medium of claim 19, wherein the instructions cause the one or more processors to:
discriminate between two or more different arrhythmia types based at least in part on whether the relative time of occurrence of the predetermined feature falls within a defined time window of the cardiac cycle duration, wherein the defined time window has a duration that is less than the cardiac cycle duration.
